(19) United States
(12) Patent Application Publication
(10) Pub. No.: US 2006/0195342 A1
(43) Pub. Date: Aug. 31, 2006

(54) Method and system for providing medical healthcare services

(76) Inventors: Mansoor Khan, Westboro, MA (US); Ahmad Kasmieh, Hollis, NH (US); Fauzia M. Khan, Westboro, MA (US)

Correspondence Address: IANDIORIO & TESKA, 260 BEAR HILL ROAD, WALTHAM, MA, 02451-1018, US

(21) Appl. No.: 11/292,434

(22) Filed: Dec. 2, 2005

Related U.S. Application Data
(63) Continuation-in-part of application No. 10/384,511, filed on Mar. 7, 2003.
(60) Provisional application No. 60/363,015, filed on Mar. 8, 2002.
(60) Provisional application No. 60/443,305, filed on Jan. 29, 2003.
(60) Provisional application No. 60/632,269, filed on Dec. 2, 2004.

(51) Int. Cl. G06F 19/00; A61B 5/00

(52) U.S. Cl. 705/3; 600/300

(57) ABSTRACT

A method is provided for ordering over a network one or more tests for a medical condition for a patient, the method including the steps of providing to the user over the network one or more tests for the patient that can be selected, allowing a user to select over the network one or more tests, determining whether a constraint exists on ordering any of the selected tests; ordering the selected tests over the network, obtaining a result of each of the ordered tests, and providing an automated evaluation based upon feedback resulting from the ordered tests. Using the methods and systems described, a user, such as a physician, can easily obtain information over a network about a large number of tests and order any of the tests over the network. The server can also obtain payment and related information from the user at the time the one or more tests are ordered.

### RELATED APPLICATIONS

This application claims benefit of U.S. Provisional Application Ser. No. 60/632,269, filed on Dec. 2, 2004, entitled “Managing Lab Testing Services Utilization” and is a continuation-in-part application of U.S. Ser. No. 10/384,511, filed Mar. 7, 2003, entitled “Method and System for Providing Medical Health Care Services”, by Khan et al. which claims benefit of U.S. Provisional Application Ser. No. 60/363,015, filed Mar. 8, 2002, entitled “Method for Diagnosing a Problem”, and claims benefit of U.S. Provisional Application Ser. No. 60/443,305, filed on Jan. 29, 2003, entitled “A Method for Creating and Implementing Standards and Guidelines for Medical Diagnosis and Therapeutics”, all applications of which are herein incorporated by reference.

### FIELD OF THE INVENTION

This invention relates to a method and system for providing medical healthcare services and products, and more specifically, a method and system for ordering procedures for medical healthcare services.

### BACKGROUND OF THE INVENTION

Recent changes in the medical healthcare industry have resulted in a substantial increase in the number of laboratory tests that are available today to a practicing physician. Examples of these tests include genomic sequencing, gene expression profiling, and tests that relate to bioterrorism and bioinformatics. As many as 9,000 tests may exist, but typical physicians may only be aware of a small percentage of these tests. To make matters even more difficult for physicians, new tests and new standards of care are being created at an explosive rate. Thus, there exists a need to present this vast amount of information about tests and procedures to physicians in an effective and reliable manner.

At the same time that the number of tests and procedures are increasing, healthcare institutions are experiencing cost constraints due to managed care reimbursement programs and thus would like to exercise caution while ordering laboratory tests, especially if the tests are outsourced to an external laboratory. Healthcare institutions would also like to ensure that they receive payment for the tests that are ordered. This is an important consideration since as much as eighty percent of the tests that insurance do not cover are never paid for by a patient. Thus, laboratorians, clinicians and medical compliance specialists would benefit in taking a proactive role in developing utilization guidelines and clinical standards when ordering tests or procedures. Similarly constraints and guidelines need to be applied to procedures and therapeutics.

### BRIEF SUMMARY OF THE INVENTION

It is therefore an object of this invention to provide a medical healthcare services system that recommends one or more appropriate tests as well as procedures and medications to a physician based upon a provisional diagnosis.

It is a further object of this invention to provide such a medical healthcare services system that automatically orders each of the tests, procedures or medications selected by a physician.

It is a further object of this invention to provide such a medical healthcare services system that determines if any constraints exist on ordering any of the tests, procedures or medications selected by a physician.

It is a further object of this invention to provide such a medical healthcare services system that obtains payment from the user for tests, procedures or medications that are ordered and are not covered by insurance.

It is a further object of this invention to provide such a medical healthcare services system that provides an evaluation based upon feedback resulting from the ordered tests, procedures or medications.

The invention results from the realization that a more effective healthcare services system can be obtained by providing to a user over a network one or more tests or procedures for a patient that can be selected, allowing a user to select one or more of the tests or procedures, determining whether there is any constraint on any of the selected tests/procedures, ordering the selected tests/procedures over the network, obtaining a result of each of the ordered tests/procedures, and providing an evaluation based upon feedback resulting from the ordered tests/procedures. One of the procedures may include ordering medication. Also a therapy may be recommended based upon the results of the tests and/or procedures.

This invention features a method for selecting over a network one or more tests for a medical condition for a patient, the method including providing to the user over the network one or more tests for the patient that can be selected, allowing the user to select over the network one or more tests, determining whether a constraint exists on ordering any of the selected tests, ordering the selected tests over the network, obtaining a result of each of the ordered tests, and providing an automated evaluation based upon feedback resulting from the ordered tests.

In one embodiment, the method further may further include providing a report based upon the automated evaluation. The step of determining whether a constraint exists may include evaluating whether a different lab or radiology test is preferable or whether a drug the patient is currently taking will cause an interaction with the selected test. The method may further include the step of determining whether to start or stop a drug, to select drug alternatives, or to change a drug dosage. The step of providing an evaluation may include providing an evaluation of the one or more tests provided to the user, an evaluation of the constraints of diagnostic algorithms, or an evaluation of the user. The feedback may be obtained from the users or from outcome data of the ordered tests. The method for ordering over a network one or more tests for a medical condition for a patient may further include the steps of recommending to the user over the network one or more tests based on a provisional diagnosis of the condition, and providing to the user over the network an analysis of the one or more recommended tests if more than one test is recommended. The method may further include the steps of recommending over the network at least one treatment based on the test results to cure the condition, identifying whether there is any constraint on ordering the recommended treatment, and ordering the treatment if no constraint exists for user to obtain the treatment in which the treatment is selected from a medical or surgical procedure, one or more drugs, or a combination thereof. Each of the constraints may have a plurality of levels of significance. The method may further include the step of allowing the user to provide an override to order the test if a constraint exists on ordering the test and may also include the step of providing a notification of the override to one or more parties. The step of determining whether any constraints exist may include comparing a code for the provisional diagnosis with a code for each of the selected tests to determine whether there is any constraint on each of the selected tests. The method may also include the step of providing to the user a cost analysis of each of the tests if more than one test is recommended. The user may be a physician. The step of determining whether any constraints exist may include determining whether a test meets guidelines, is ordered too frequently, is obsolete, is FDA approved, is too expensive, or would be ineffective due to a drug the patient is taking. A constraint may be selected from the group of causes of increased follow-up visits, a drug the patient is taking, a drug the patient is not taking, the result of a screening test, an indication or the lack of an indication from a test result or prior diagnosis.

This invention further features a method for selecting over a network one or more tests for a condition for a patient, the method including the steps of allowing a user to select over the network a first test for the patient, determining whether a constraint exists on ordering the selected first test, recommending to the user over the network one or more secondary tests if a constraint exists to obtain the selected first test, allowing the user to select over the network one of the secondary tests, ordering the selected secondary test over the network, and obtaining a result of the selected secondary test.

In one embodiment, the step of determining whether a constraint exists may include evaluating whether a test meets guidelines, is ordered too frequently, is obsolete, is FDA approved, is too expensive, or would be ineffective due to a drug the patient is taking. The method may further include the steps of recommending to the user over the network one or more initial tests based on a provisional diagnosis of the condition, and providing to the user over the network an analysis of the one or more recommended initial tests if more than one initial test is recommended. The method may also include the step of allowing the user to provide an override to order the selected first test if the test is not covered by insurance.

This invention further features a system for providing medical healthcare services, including a computer readable medium having computer readable program code for ordering over a network one or more tests for a condition, the computer readable program code executable on a computer system and including instructions for causing the computer system to allow a user to select over the network one or more of the tests, causing the computer system to determine whether a constraint exists on ordering any of the selected tests, causing the computer system to order the selected tests over the network, causing the computer system to obtain a result of each of the selected tests, and causing the computer system to provide an evaluation based upon feedback resulting from the ordered tests.

In one embodiment, the computer readable program code may further include instructions for causing the computer system to obtain payment for a test if a constraint exists on ordering the test, to provide one or more final diagnoses for the condition based on the result of each of the selected tests, to allow the user to provide over the network an override to order the test if a constraint exists on ordering the test, and to provide a notification of the override to one or more parties. The computer readable program code for causing the computer to provide an evaluation based upon feedback may further include computer readable program code for causing the computer system to provide an evaluation of the user or the one or more tests provided to the user, to evaluate whether a drug the patient is currently taking will cause an interaction with the selected test, and to provide an evaluation of the one or more tests provided to the user.

This invention further features a server for providing medical healthcare services, the server including a computer including a processor and computer readable program code executable on the processor for ordering over a network one or more tests for a condition, the computer readable program code configured to allow a user to select over the network one or more of the tests, determine whether a constraint exists on ordering any of the selected tests, order over the network the selected tests, obtain a result of each of the selected tests, and provide an evaluation based upon feedback from the ordered tests.

In one embodiment, the computer readable program code may be further configured to obtain payment for a test if a constraint exists on ordering the test, to allow the user to provide an override to order a test if a constraint exists on ordering the test, to provide a notification of the override to one or more parties, to cause the computer system to provide a notification of the override to one or more parties. The computer readable program code for causing the computer to provide an evaluation based upon feedback may include computer readable program code for causing the computer system to provide an evaluation of the one or more tests provided to the user, for causing the computer system to evaluate whether a drug the patient is currently taking will cause an interaction with the selected test, and for causing the computer system to provide an evaluation of the one or more tests provided to the user. The server may further include a database that includes information about patients' historical data or information about decision support guidelines. The computer readable program code may be further configured to collect user feedback, and modify the recommended tests based upon the user feedback.

This invention also features a method for selecting over a network one or more procedures for a medical condition for a patient, the method including providing to the user over the network one or more procedures for the patient that can be selected, allowing the user to select over the network one or more procedures, determining whether a constraint exists on ordering any of the selected procedures, ordering the selected procedures over the network, obtaining a result of each of the ordered procedures, and providing an automated evaluation based upon feedback resulting from the ordered procedures.

In one embodiment, the step of determining whether a constraint exists may include the step of reviewing a plurality of rules. The rules may be divided into categories of rules. The step of reviewing the plurality of rules may include reviewing only some of the categories of rules to expedite the step of determining whether a constraint exists.

### BRIEF DESCRIPTION OF THE DRAWINGS

Other objects, features and advantages will occur to those skilled in the art from the following description of a preferred embodiment and the accompanying drawings, in which:

FIG. 1 is a schematic block diagram of a medical healthcare services system in accordance with the present invention;

FIG. 2 is a more detailed schematic block diagram of the medical healthcare services system of FIG. 1;

FIG. 3 is a flowchart of a method for ordering one or more tests for a condition using the medical healthcare services system of FIG. 1;

FIG. 4 is a more detailed flowchart of the method of FIG. 3 for ordering one or more tests;

FIG. 5 is another embodiment of a method for ordering one or more tests that uses the medical healthcare services system of FIG. 1;

FIG. 6 is yet another embodiment of the method of FIG. 3 in which user feedback is collected and used;

FIG. 7 is an exemplary software interface for a software program used on a server of the medical healthcare services system of FIG. 1;

FIG. 8 is a software program interface that shows information about a specific test for the program of FIG. 7;

FIG. 9 is a software program interface that shows a shopping cart having two ordered tests for the program of FIG. 7;

FIGS. 10a and 10b are a software program interface that shows the payment acceptance means for the program of FIG. 7;

FIG. 11 is an exemplary medical healthcare algorithm that is used with the software program of FIG. 7;

FIG. 12 is an exemplary software program interface that shows a patient profile used with the software program of FIG. 7;

FIG. 13 is an exemplary software program interface that shows patient clinical history for the software program of FIG. 7;

FIG. 14 is an exemplary software program interface that shows the patient medical history for the software program of FIG. 7;

FIG. 15 is an exemplary software program interface that shows a patient's anatomical pathology reports for the software program of FIG. 7;

FIG. 16 is an exemplary software program interface that shows a patient's clinical pathology reports for the software program of FIG. 7;

FIG. 17 is an exemplary software program interface that allows a user to order tests for the software program of FIG. 7;

FIG. 18 is a schematic block diagram of a general embodiment of the subject invention;

FIG. 19 is a table that shows examples of benefits from the use of constraints as shown in FIGS. 3-6 and 18;

FIG. 20 is a screen shot that shows information about utilization management guidelines after constraints have been applied to a selected test;

FIG. 21 is a schematic block diagram of another embodiment of the medical healthcare services system of FIG. 2 in which a patient historical database is located at the site of the system operator;

FIG. 22 is a schematic block diagram of yet another embodiment of the medical healthcare services system of FIG. 2 in which the patient historical database is located off-site from the site of the system operator; and

FIG. 23 is a schematic diagram of the constraint rules divided into categories.

### DISCLOSURE OF THE PREFERRED EMBODIMENT

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings.

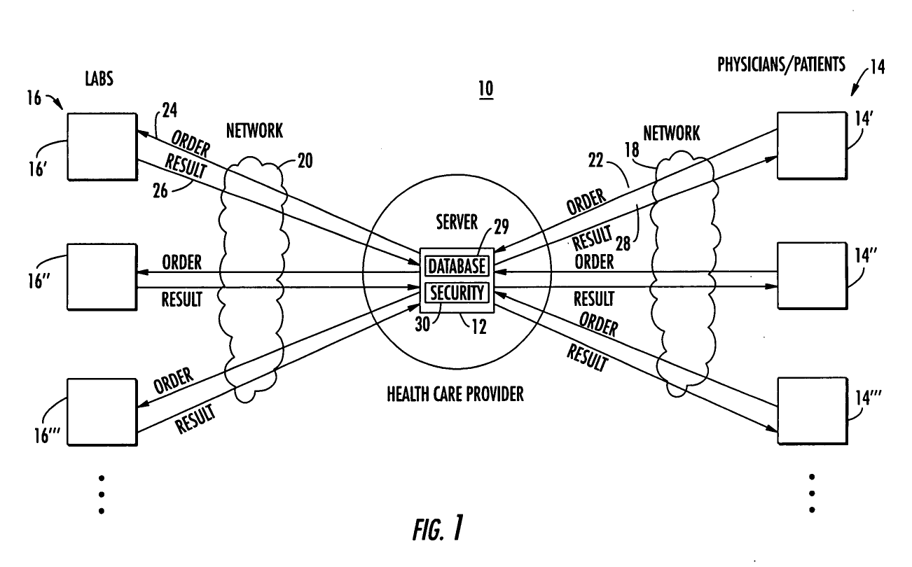

There is shown in FIG. 1 a medical healthcare services system 10 which includes a server 12, one or more user terminals 14, one or more lab terminals 16, and networks 18 and 20. User terminals 14 can include one or more remote or local terminals 14′, 14″, and 14′″ . . . , each of which can be any apparatus that can connect to server 12 through network 18, such as a computer, computer terminal, cell phone, personal digital assistant (PDA), tablet PC, mobile phone, a programmable user interface, a programmable application interface (API) that achieves electronic data interchange, or any apparatus with a web browser. Lab terminals 16 can include one or more remote or local lab terminals 16′, 16″, 16′″, each of which can include any apparatus that can connect to the server through network 20, similar to user terminals 14. Server 12 is preferably located at a healthcare provider's location, such as a hospital, but alternatively can be located at a lab or other remote location or even split amongst different physical locations.

Typically, a user, such as a physician, obtains information or places an order for a test or procedure over terminal 14′ through line 22 which connects to server 12. A procedure as described herein can include a test, a lab procedure, a radiology procedure, the ordering of medication, a therapy, or a medical or surgical procedure. If the physician places an order for a test or procedure, this information is transmitted over line 24 through network 20 to a lab with one of lab terminals 16. Once a lab has completed a test or procedure in accordance with the physician's instructions, the results of the test or procedure are transmitted over line 26, which can be the same line as line 24, to server 12. The information relating to the result of the test or procedure is transmitted back to the physician over line 28, which can be the same line as line 22. In this manner, a physician is able to obtain information about a patient, obtain information about a test or procedure, and/or order a test or procedure from a lab. Lab terminal 16′ receives the test or procedure order from the physician and transmits the result back to server 12 so that the physician can use the result of the test or procedure to help properly diagnose a patient's condition. Also, a physician at terminal 14′ can communicate with another physician at terminal 14″ to obtain information such as a second or an expert opinion. The communication lines 22, 24, 26, 28, etc. described herein can be either a wire or wireless communication line.

Server 12 typically includes or has access to one or more databases 29 which store information about each patient including history and diagnosis, the tests and/or procedures and results thereof relating to each patient, as well as information relating to each of the tests or procedures that a physician can order. Server 12 also includes one or more security programs 30 to ensure that physicians, patients, and labs only have access to the data for which they have been given appropriate authorization.

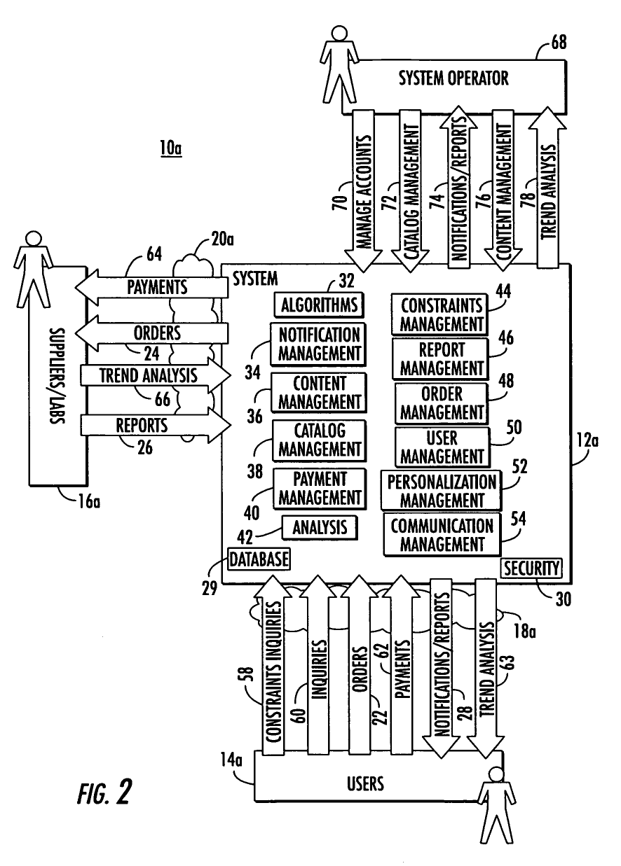

In a more specific embodiment, as shown in FIG. 2, where like parts have been given like numbers accompanied by a lower-case “a”, medical healthcare services system 10a includes a server 12a that is accessed by users 14a, labs or suppliers 16a, or a system operator at terminal 68. Server 12a includes a number of software programs including an algorithms program 32, a notification management program 34, a content management program 36, a catalog management program 38, a payment management program 40, an analysis program 42, a constraints management program 44, a report management program 46, an order management program 48, a user management program 50, a personalization management program 52, and a communication management program 54. Although the programs on server 12a are described herein as being separate programs, it should be understood that any or all of these programs could be combined into any number of programs or could be used separately as described on server 12a.

Algorithms program 32 contains information that relates symptoms and/or diagnoses to specific tests, procedures and/or drugs. Using algorithms program 32, server 12a assists physicians in providing provisional and/or final diagnoses relating to a patient's symptoms and also provides a physician with recommended tests, procedures and/or drugs that correspond to a provisional, differential or a final diagnosis. Notification management program 34 provides to physicians or patients information relating to a provisional, differential or a final diagnosis or to specific tests, procedures and/or drugs. With notification management program 34, server 12a can provide alerts to users, such as physicians or patients, regarding, for example, new information, new tests that help test for a specific diagnosis, or new treatments relating to a specific diagnosis and alerts related thereto. A treatment as described herein refers to the application or cessation of a treatment, therapy, drug, or a medical or surgical procedure, or a combination thereof.

Content management program 36 maintains and provides information relating to each specific test, procedure and/or drug. For example, information required for each test or procedure can include the information necessary to complete the test or procedure, the specimens to be collected for the test, how information and collected items are to be sent to a specific lab, and the estimated turn-around time for a specific test. Payment management program 40 controls the processes necessary to obtain payment for a specific test or procedure. A patient may accept responsibility for payment through executing an ABN and provide payment at a later time. A patient may alternatively provide payment through other methods including a credit card. For example, payment management program 40 can control how payment is accepted, when payment is accepted, whether or not any credit limit exists for a particular test or procedure, and any other processes necessary to obtain and track payment for any test.

Analysis program 42 is used to provide analyses of tests or procedures and to analyze trends that relate to prior tests or procedures that were ordered by physicians. Analysis program 42 also provides the user with an analysis, such as a cost, cost/benefit, or turn-around time analysis of each test or procedure if the server recommends more than one to the user. Analysis program 42 can also use data mining to obtain the information relating to trends. For example, analysis program 42 can compare the actual turn-around times with estimated turn-around time to modify the estimated turn-around time if necessary. Analysis program 42 can be used to analyze the efficacy of the test or procedure as it relates to the condition. Analysis program 42 can also compare test or procedure results that it obtains with results of the general population to ensure that the test or procedure results obtained from a specific lab are within reasonable limits. For example, if cholesterol tests from a specific lab show cholesterol levels that are far higher than in the cholesterol tests of the general population, analysis program 42 could determine that the cholesterol tests obtained from the specific lab are potentially inaccurate. Additionally, analysis program 42 can also analyze each physician's performance.

Constraints management program 44 includes guidelines as to whether or not a physician can order a test or procedure for a particular patient or in general. Constraints management program 44 not only provides information about what tests or procedures cannot be ordered but also about what tests or procedures may be preferable to order. For example, constraints management program 44 may recommend that a physician order a new test or procedure that was not recommended previously. The guidelines used by constraints management program 44 can be derived from medical programs such as Medicare, can be derived from insurance programs that dictate which tests or procedures can or cannot be ordered for specific patients or healthcare programs, by clinical evidence or by the user or healthcare institution, possibly using data derived from analysis program 42. Constraints management program 44 also determines if a patient has previously undergone a specific test or procedure and therefore does not need to have the test or procedure completed again. Constraints management program 44 can also use existing diagnoses, test results and patient statistics to recommend a test, procedure, and/or drugs.

Report management program 46 generates reports that include information about specific patients and/or reports about specific physicians, and determines who can view these reports. For example, report management program 46 may determine that one physician or healthcare provider has authorization to review a specific report but that another healthcare provider does not. Order management program 48 maintains and provides information that needs to go to one or more groups or systems, such as labs, clinical systems or financial systems, which can include the ability to route necessary information to the one or more groups or systems. Order management program 48 can route information based upon considerations such as a lab's ability to do a test, turn-around time or cost.

User management program 50 is a roles-driven user management program. User management program 50 defines roles for each user of healthcare management system 10a and assigns privileges to each of the roles. A user, such as a patient, may not have any privileges to access server 12a, but may still be assigned a role by user management program 12a. The roles assigned to each user assist security program 30 in determining if a specific user can access specific information on server 12a.

Personalization management program 52 presents each user that accesses the server 12a with a personalized user interface. Personalization management program 52 can personalize each user's personalized interface on the fly such that a user has the most up-to-date information available to the user. Personalization management program 52 can provide, for example, data relating to the effectiveness of specific tests or procedures that the user has taken, ordered, or may order in the future.

Communication management program 54 allows communications between various users of the medical healthcare services system 10a. For example, communication management program 54 can allow the communication between two physicians who wish to communicate about a specific patient. Also, the communication management program can allow communication between a physician and a lab regarding a specific test.

As described above, each of users 14a orders a test or procedure over line 22 and receives test/procedure results, a notification or a report on line 28. Additionally, users 14a can make an inquiry with regard to whether there are any constraints with respect to a particular test, procedure and/or drug on line 58. Each of the users 14a can also make a general inquiry with regard to one of the patients or a specific test or procedure on line 60. Payment for a test or procedure is transmitted from the user to a laboratory over line 62.

Also as noted above, each of the labs 16a can receive an order on line 24 and transmit test or procedure results or a report over line 26. Additionally, each of the labs 16a receives payment for a test or procedure or other service or product on line 64 and provides trend analyses on line 66. It should be noted that each of the communication lines between labs 16a and server 12a, as well as the communication lines between users 14a and server 12a, can either be communicated over a single data line or several data lines as shown.

At terminal 68, a system operator, such as a server administrator or information technology personnel, manages the information and programs on server 12a. The system operator manages accounts on line 70, updates or manages catalogs and catalog management on line 72, and manages the content in the content management program on line 76. The system operator receives notifications or reports on line 74 and receives trend analyses on line 78. It should be noted that all the data lines between the server 12a and system operator terminal 68 can be either individual communication lines or can be a single communication line.

Additionally, system 10a stores, organizes and provides medical knowledge and standards to issue recommendations to care providers and patients. The data elements stored, organized and provided by system 10a include pre- and post-encounter check lists which may be region/group/institution/patient specific. These encounters may be doctor specific patient visit lists, pre-admission, pre-surgical, post encounter, post surgical, discharge, chronic care sheet and any other embodiments of care-provider—patient contact. Data elements may also include care rule scheduler, clinical rules regarding care/tests/drug/procedure, response to the care provider, any limitations or contraindications to the response, frequency and severity of response. These alerts have different levels of clinical impact and urgency. Care recommendations may be controlled using a volume dial at different levels.

Other data elements stored, organized and provided by system 10a include information about tests such as tests for laboratory cause of increased value, causes of decreased value, spurious value, reference range specific for age and sex. Clinical Notes and rules that define the clinical relevance of the test. It also includes critical values which determine the severity or stratification of these alerts. Also stored, organized and provided is the compliance information for billing as well as associated drugs that influence the test. Other stored, organized and provided information includes frequency, associated diagnoses, other relevant tests, test panels, follow up testing, and indications for tests.

Drug data information stored, organized and provided by system 10a includes drug, group generic, proprietary name, code, indications, route of administration and dosage, allergy check, drug interactions, recommended tests/panels for starting, stopping, transitioning to another route of administration or monitoring of drugs. Alternate drug suggestions based on any of the earlier data elements and in addition diagnosis, cost, any patient/group/institution/insurer specific information or laboratory date. Alerts based on similar sounding drug names may be another element.

System 10a also provides care alerts/recommendations that may include, but are not limited to, patient diagnosis and symptom codes, patient demographics and status including age, sex, weight, vital signs, state, etc. The care alerts/recommendations provided by system 10a include the severity of alert, recommended care, not recommended care, message to care provider, test, medications, care recommendations as well as strength/level/type of evidence data for issuing the recommendation and any image and references relevant to the tests. These alerts may be issued to patients as well.

System 10a also provides an associated web service that can track historical information regarding medical guidelines and standards used by a group or an institution. This service provides a way or means to gather information from feedback and data mining into the existing standards to develop new group/institution specific standards.

Other data elements stored, organized and provided by system 10a include a patient list with alerts and recommendations for a physician to review in anticipation of a scheduled office visit or an unscheduled call. Alternatively data mining of individual patient data can lead to scheduling of a visit or consultation in person or through the web or the phone or any other way. These care alerts are based upon but are not limited to the following data elements: care rule itself regarding clinical care/tests/drugs/procedures/associated codes, response to the care provider, any limitations or contraindications to the response, frequency and sensitivity of responses. These limitations may be based on present or previous diagnoses or test results, other drugs, lack of relevant information or any other factor that could impact care delivery including non-medical information such as cost, or non-availability.

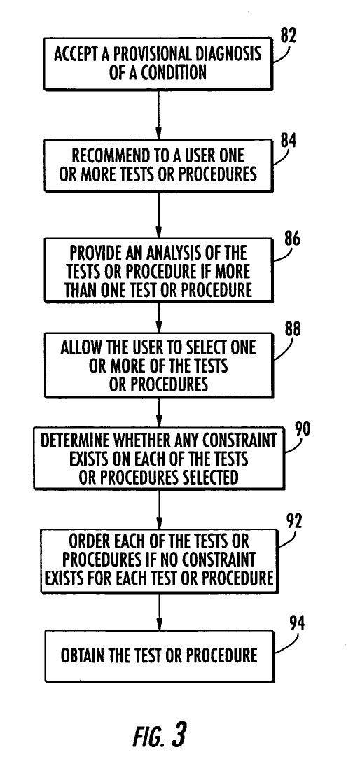

The flowchart of FIG. 3 describes ordering one or more tests or procedures and begins at step 82 with accepting a provisional diagnosis of a condition. At step 82, a user inputs into the medical healthcare server 12, FIG. 1, a provisional diagnosis from a terminal 14′. Alternatively, a user communicates to the server one or more symptoms relating to a condition and the server will provide the provisional diagnosis. In step 84, FIG. 3, the server recommends to the user one or more tests or procedures relating to the provisional diagnosis of the condition. In step 86, the server provides an analysis to the user of the tests or procedures if recommended. The analysis provided to the user can be, for example, a cost, cost/benefit or a user feedback analysis of one or more of the tests or procedures. With this information, a user, such as a physician, can make an informed choice as to which tests or procedures, if any, to order for a specific patient. In step 88, the user is allowed to select one or more of the tests or procedures.

In step 90, the constraints management program 44, FIG. 2, determines whether any constraint exists on each of the tests or procedures selected. As noted above a constraint can exist with regard to a healthcare program, a Medicare program or a user may have already ordered or taken one of the selected tests or procedures. The constraints may be clinically relevant based upon medical evidence such that an ordered test, procedure or drug is related to existing diagnoses and test results. A constraint can also include causes of increased follow-up visits, a drug the patient is or is not taking, the result of a screening test, or medications, or the lack thereof of something within a test result or prior diagnosis. In step 92, FIG. 3, the server orders each of the tests/procedures if no constraint exists for each particular test or procedure. If a constraint does exist for a particular test or procedure, the server can additionally ask the user whether the user desires to pay for the test or procedure himself. For example, the server or the healthcare provider can ask a patient to fill out an advanced beneficiary notice (ABN) which states that the patient will pay for any test or procedure that is not covered by insurance and/or that the patient recognizes that insurance will not pay for the test or procedure. In step 94, the server obtains the selected tests/procedures that were ordered. The information obtained from the selected tests or procedures can be incorporated into a corresponding patient profile data file that relates to each patient.

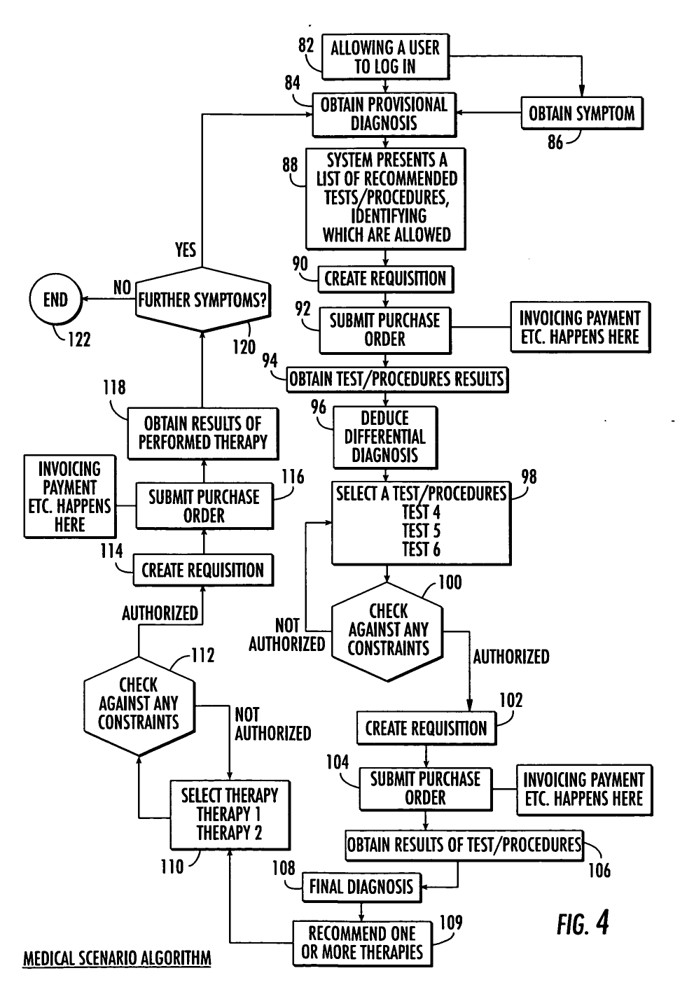

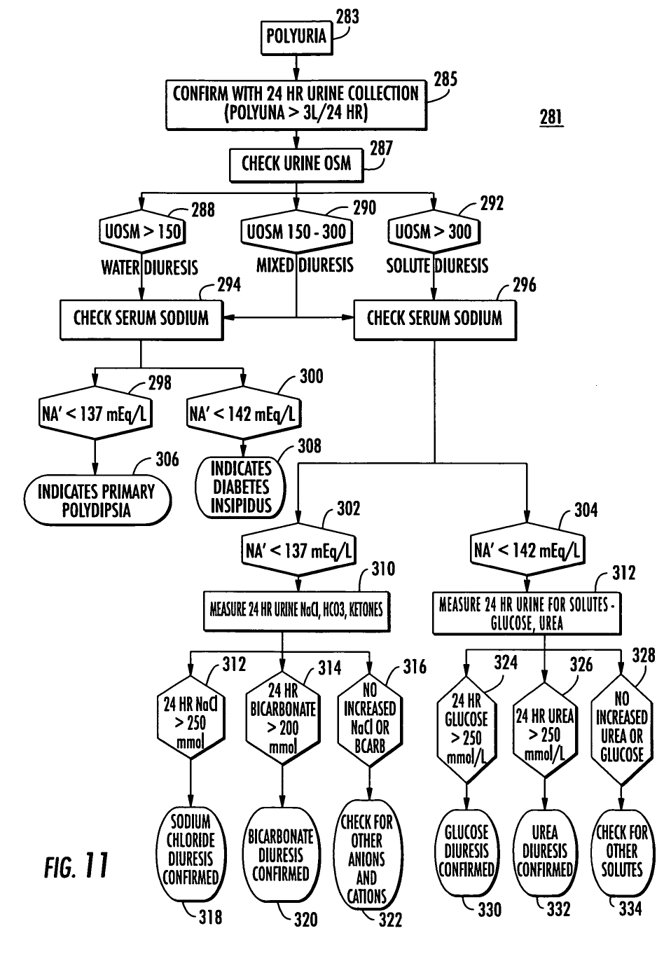

The flowchart of FIG. 4 describes ordering one or more tests or procedures and begins at step 82, with allowing a user to log into medical healthcare server 12, FIG. 1. The user may also obtain a patient profile and clinical history from a server database at step 82. In step 84, FIG. 4, the server obtains a provisional diagnosis of a condition. A user, such as a physician, provides the provisional diagnosis to the server or alternatively the server determines the provisional diagnosis after the user inputs, in step 86, one or more symptoms of the condition into the server. In step 88, the server presents a list of recommended tests/procedures to the user. An algorithm, such as the one shown in FIG. 11, can be used to determine one or more appropriate tests or procedures to recommend to a user. Alternatively, a look-up table could provide recommended tests or procedures for a condition or symptom. At this time, the server can identify which tests/procedures are allowed and do not have a constraint or the server can identify which tests/procedures have a constraint after the user has selected the tests or procedures. In step 90, the server creates a requisition for each selected test or procedure. In step 92, the server submits a purchase order to the appropriate labs at which the test or procedure will be performed. Obtaining payment from the user if necessary also occurs in step 92.

In step 94, the server obtains the results of each of the tests/procedures that were performed. In step 96, the server determines a differential diagnosis and may make a recommendation based upon the test/procedure results obtained in step 94. In step 98, the user, such as a physician, can select one or more tests or procedures in conjunction with the diagnosis determined in step 96. In step 100, the server determines whether there are any constraints against obtaining one of the tests or procedures selected in step 98. For example, the server can compare an ICD code associated with the diagnosis with the CPT code that is associated with each selected test/procedure to determine whether there is any constraint on any of the selected tests/procedures. Other codes, look-up tables or other information could be used to determine if any constraints exist. If there is a constraint at step 100 against the user ordering one of the selected tests or procedures, the user can be allowed the option to select one or more alternative tests or procedures in step 98. If there are no constraints against the one or more selected tests/procedures selected in step 98, the server creates a requisition for the one or more tests/procedures in step 102 and submits a purchase order to the appropriate labs in step 104. Obtaining payment from a user if necessary also occurs in step 104.

Once the selected tests/procedures have been completed by the appropriate labs, in step 106 the server obtains the results of the tests/procedures. The server uses the results of the test or procedure to create a final diagnosis in step 108. In step 109, the server can recommend to a user one or more therapies that correspond to the final diagnosis determined in step 108. In step 110, the user selects one or more of the therapies. A therapy as described herein can also include a procedure or treatment. In step 112, the server checks to see if there are any constraints that would restrict the user from obtaining the one or more selected therapies. For example, the server can check the ICD code associated with the final diagnosis to determine if the user can obtain each selected therapy. If the user is not authorized to obtain a specific therapy, the server allows the user to reselect one or more therapies related to the final diagnosis in step 110. The server can allow the user to provide an override to order the one or more therapies. If each of the selected therapies does not have any constraints against obtaining the therapy, the server creates a requisition for the selected therapy in step 114. In step 116, the server will submit a purchase order to one or more service providers or product suppliers for the recommended therapy. Obtaining payment from the user if necessary occurs in step 116.

In step 118, the server obtains the results of the performed therapy, which may have been performed by either a physician or the patient. In step 120, the server determines whether or not there are any further symptoms of the condition. Steps 118 and 120 can be combined into one step. If no further symptoms exist of the condition, then the server ends its routine in step 122. If further symptoms do exist of the condition, the server can go back to step 84 to determine a provisional diagnosis of the condition.

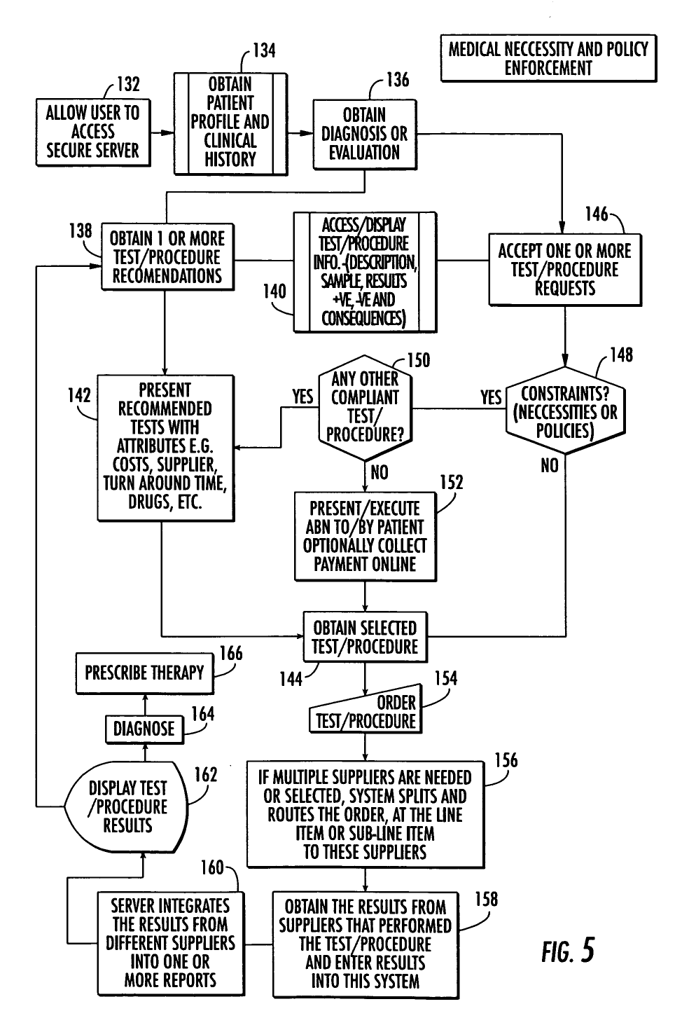

The flowchart of FIG. 5 describes obtaining one or more tests or procedures from a plurality of labs and begins at step 132 with allowing a user to access secure server 12, FIG. 1, from a terminal 14′. In step 134, FIG. 5, the server obtains a patient profile and corresponding clinical history from the server database. In step 136, the server obtains a diagnosis from the user or determines a diagnosis based upon one or more symptoms provided by the user. In step 138, the server provides one or more test/procedure recommendations based upon the diagnosis. In step 140, the server accesses and displays to the user corresponding test/procedure information, which can include the test or procedure description, a specimen of the test or procedure, sample test or procedure results, the meaning of positive and negative test or procedure results and any consequences of the results of the test or procedure. In step 142, the server presents the recommended tests/procedures to the user with the corresponding information relating to the test/procedure, which can include the information accessed in step 140 and other information such as the costs, supplier and the turn-around time of each of the one or more recommended tests or procedures. If the server recommends more than one test or procedure, the server provides to the user at step 142 an analysis of the one or more tests or procedures. In step 144, the server obtains from the user the one or more tests or procedures selected by the user and the selected supplier of a test or procedure if a test or procedure can be ordered from more than one supplier.

Alternatively, rather than using the method starting at step 138 and proceeding to step 144 in which the server recommends one or more tests or procedures, the server can accept one or more test or procedure requests from the user in step 146. The server accesses the corresponding test/procedure information in step 140. The server then determines whether or not the one or more tests/procedures have any constraints against them in step 148. If there are no constraints against ordering the tests or procedures, the server obtains the selected tests/procedures in step 144. If there are constraints against ordering the one or more tests/procedures selected in step 146, the server determines in step 150 if there are any other tests or procedures that do not have constraints against the user obtaining them. If there is an alternate test or procedure without constraints, the server presents the recommended test or procedure to the user in step 142. If there is no test or procedure that does not have a constraint, in step 152 the server verifies that the patient has executed an advanced beneficiary notice (ABN), which gives a recognition that insurance will not pay for the test or procedure, or that the patient has otherwise provided payment for the one or more noncompliant tests or procedures on-line. Once the server has verified the user has provided payment or has accepted responsibility for payment, the server obtains the selected tests/procedures in step 144.

In step 154, the server orders the one or more tests/procedures. If one or more suppliers or labs are required to obtain the one or more tests/procedures, in step 156 the server splits and routes the orders to the appropriate suppliers or labs. In step 158, the server obtains the test/procedure results from the one or more suppliers or labs that performed the tests/procedures and enters the test/procedure results into the server database. In step 160, the server integrates the results from the different suppliers into the one or more corresponding reports, which can be patient profiles. In step 162, the server displays the test/procedure results to a user. In step 164, the server provides a diagnosis to the user and in step 166 the server or physician prescribes a therapy based upon the diagnosis provided in step 164. At step 162 when test/procedure results are displayed, recommendations to obtain one or more tests or procedures at step 138 may also be displayed such that a user can be given an appropriate recommendation for follow up testing for the patient.

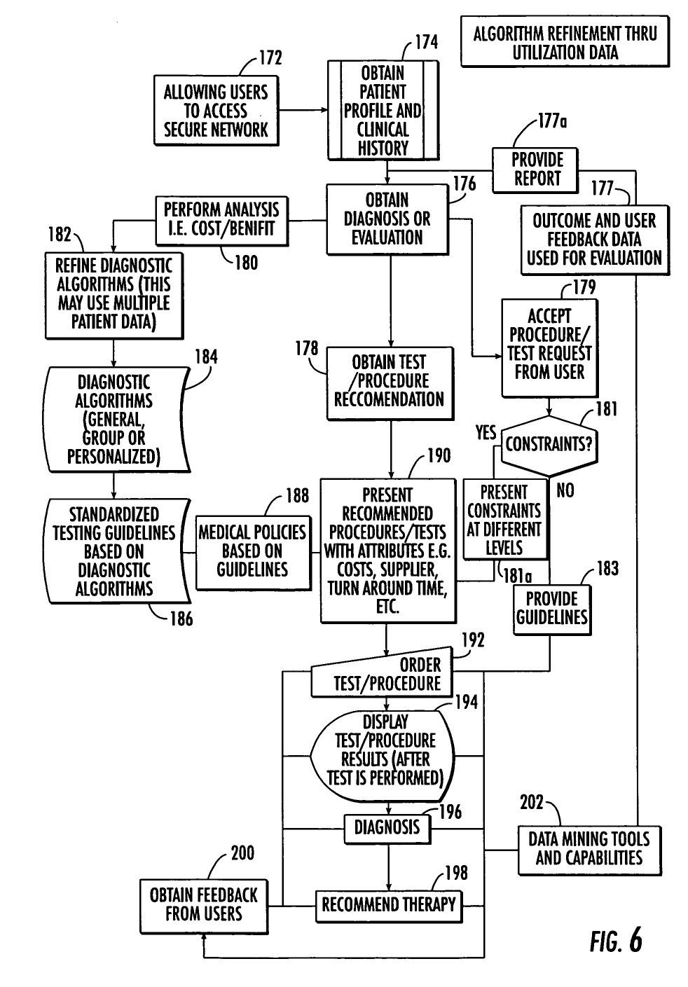

The flowchart of FIG. 6 describes a method that includes using user feedback and begins at step 172 with allowing users to access secure network 12, FIG. 1, through terminal 14′. In step 174, FIG. 6, the user can obtain a patient profile and clinical history, which describes tests/procedures that the patient has already taken and tests/procedures that may currently be on order. In step 180, the server obtains a diagnosis, which can be obtained either from the user or from an algorithm provided by the server. In step 178, the server determines which one or more tests/procedures to recommend to the user based upon the obtained diagnosis at step 176. In step 178, the server performs an analysis, such as a cost or cost/benefit analysis, of the one or more tests/procedures. In step 182, diagnostic algorithms that determine which one or more tests/procedures to recommend can be refined based upon outcome and user feedback data obtained in step 177. This feedback data may use data from multiple patients. In step 184, the server determines whether or not to revise the diagnostic algorithms for just the one person or for a group of people. In step 186, the server determines whether or not to change the standardized testing guidelines based upon the revised diagnostic algorithms in step 184. In step 188, medical policies can be revised based upon the revisions made to the testing guidelines in step 186. Steps 182-188 can be performed by the server or by the user using programs located on the server. In step 190, the user is provided with the one or more recommended tests/procedures with an analysis that includes one or more attributes of each test/procedure such as the cost of the test/procedure, the test/procedure supplier, and the estimated turn-around time of the test/procedure. The tests/procedures recommended at step 190 can be based upon the data obtained at steps 182-188.

Alternatively, rather than having the server recommend one or more tests/procedures to the user, the server can accept a test/procedure request from the user in step 179. In step 181, the server determines whether or not the one or more requested tests/procedures have any constraints against them. If there are no constraints against ordering the requested tests or procedures, the server may provide test/procedure guidelines at step 183 and it orders the tests or procedures in step 192. If there are constraints against ordering the requested tests or procedures, in step 190 the server automatically recommends one or more alternative tests or procedures to the user, but can provide the user with the option to override the system to order the one or more tests/procedures with constraints. At step 181a, the server may present the constraints in a format showing different levels of significance that relate to urgency, warnings, criticality or other information.

In step 192, the server orders the one or more tests/procedures that were selected by the user either in step 190 or step 179. Once the server obtains the test/procedure results, in step 194 the test/procedure results are displayed to the user. In step 196, the server provides to the user a diagnosis of the condition based upon the test/procedure results. In step 198, the server recommends therapy for the diagnosed condition.

In step 200, the server obtains feedback from users regarding the effectiveness or desirability of the therapy, the diagnosis or the one or more tests/procedures that they selected. The feedback obtained from the users in step 200 can be used to modify the processes of ordering a test or procedure in step 192, displaying the test/procedure results in step 194, providing a diagnosis in step 196, and recommending a therapy in step 198. Alternatively or additionally, data mining tools used in step 202 can be used to analyze the user feedback as well as the outcome data from steps 194, 196 and/or 198, which can be used for the creation, modification and the evaluation of the tests/procedures and diagnostic algorithms, standardized testing guidelines, and medical policies based upon the guidelines in steps 182, 184, 186 and 188 as described above.

As noted above, data mining tools 202 are used for evaluation at step 177. This evaluation can include an evaluation of the appropriateness of the one or more tests/procedures provided to the user, an evaluation of diagnostic algorithms, or an evaluation of the user such as a physician. The feedback used for evaluation at 177 is obtained from the users or from the outcome data of the ordered tests, procedures and therapies. The evaluation may be automated or may be manually provided. A report may be generated at step 177a regarding the evaluation provided at step 177.

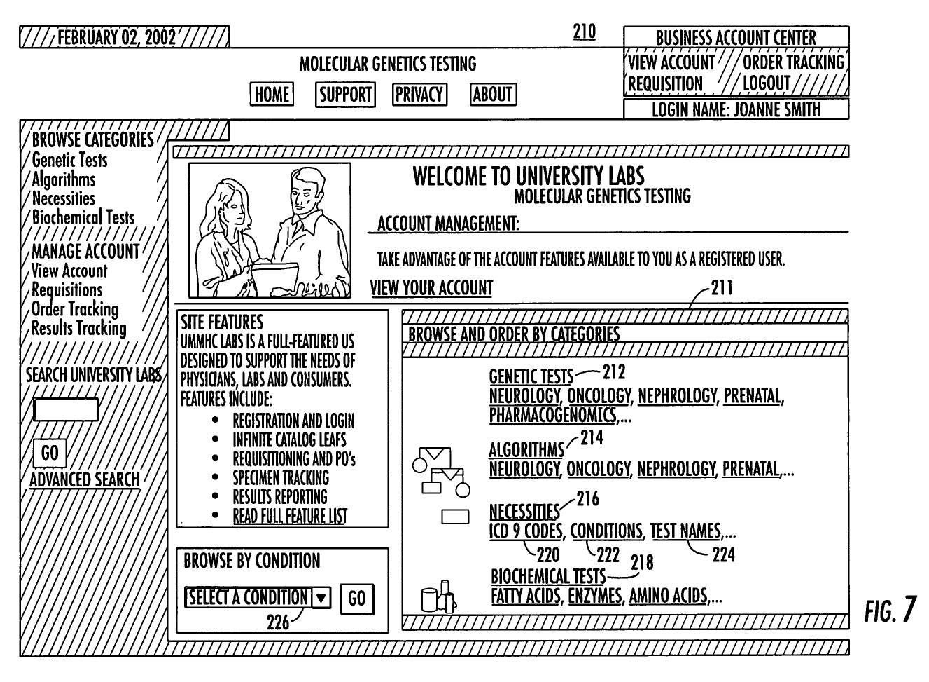

A software program interface 210, FIG. 7, includes a category list 211 having a number of links to categories that the user can click on and select to view. For example, category list 211 includes a genetic tests link 212, an algorithms link 214, a necessities link 216, and a biochemical tests link 218. Each of the category links can be further broken down into subcategory links. These may also be viewed as a tree view, general view, or algorithm view. For example, genetic tests link 212 can include links to the different types of genetic tests, algorithms link 214 can include links to the different specific types of algorithms, necessities link 216 can include a codes link 220, a conditions link 222 and a test names link 224. Biochemical tests link 218 can include subcategory links of specific types of biochemical tests. A link to anatomical pathology tests can also be provided. A user can also browse by condition by selecting button 226 and scrolling to a particular condition to view information associated with that condition.

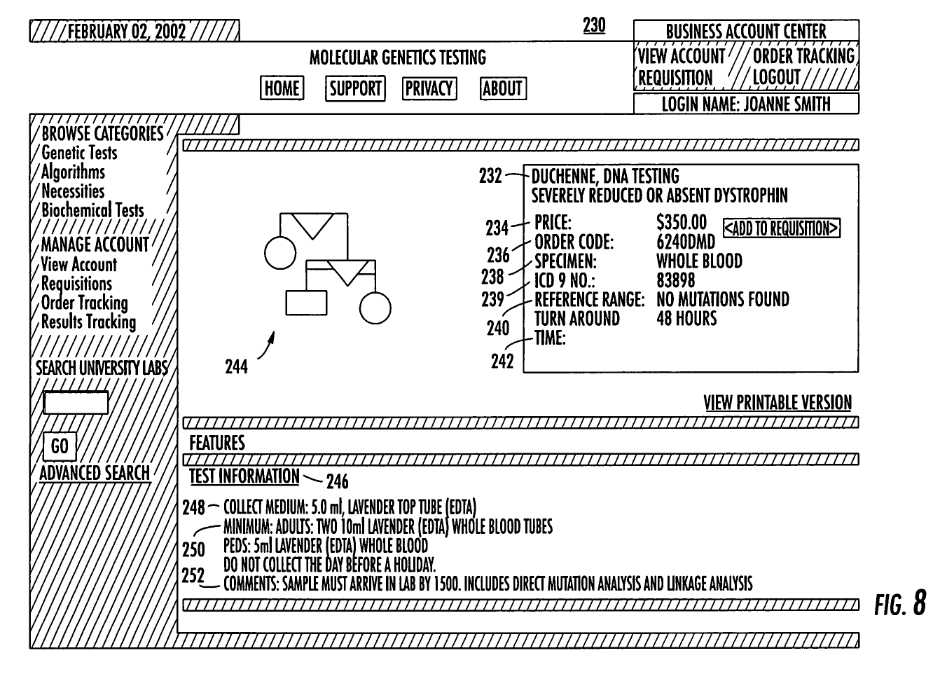

If a user selects a particular test on interface 210, software interface 230, FIG. 8, is displayed to the user. Software interface 230 includes information about a test 232, such as Duchenne DNA testing. The information shown about the test can include its price 234, the order code 236, the specimen required to obtain the test 238, one or more ICD codes 239 relating to one or more diagnoses, the reference range of the test 240 and the estimated turn-around time 242 of the test. Additionally, interface 230 includes a sample algorithm 244 associated with the tests that can be clicked on to show a larger view of the algorithm. Further test information 246 is also shown which includes the necessary collection medium 248 in which the test sample is to be collected, the minimum amount 250 of test sample to be collected, and any other comments 252 that are relevant to the administration or ordering of the tests.

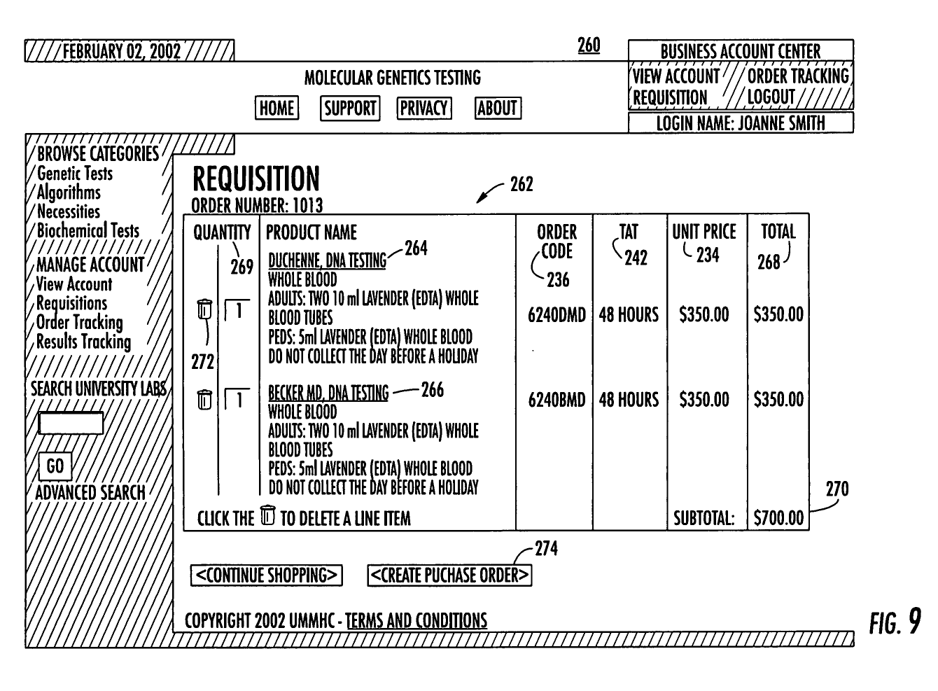

A requisition software interface 260, FIG. 9, includes a shopping cart 262 that displays the one or more ordered tests 264, 266. For each test, shopping cart 262 includes the test's order code 236, the turn-around time 242, the unit price 234 of the test and the total cost 268 which will depend on the quantity 269 of tests ordered by the user. The user can click on a symbol 272 of a trashcan to delete one of the tests from the requisition. A subtotal price 270 is displayed for the total cost of all the tests 264, 266 that are ordered. A user creates a purchase order for the test by clicking on button 274.

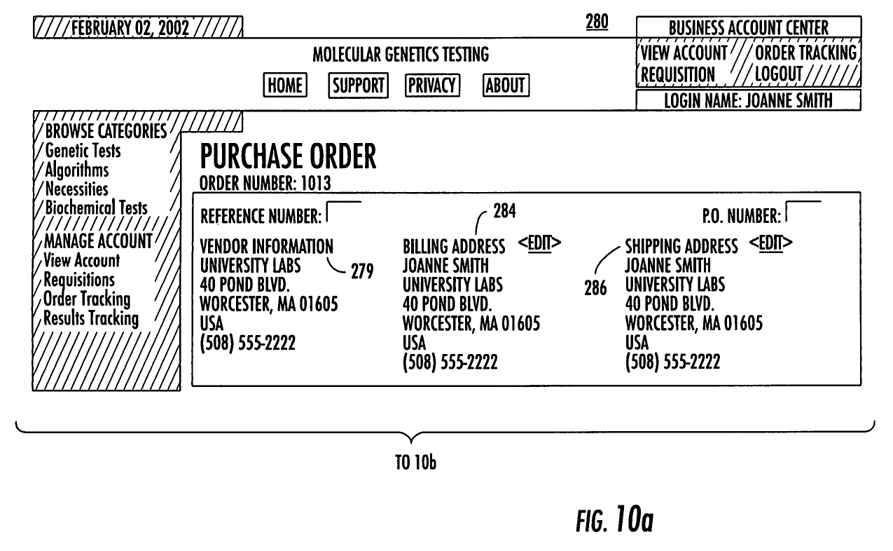

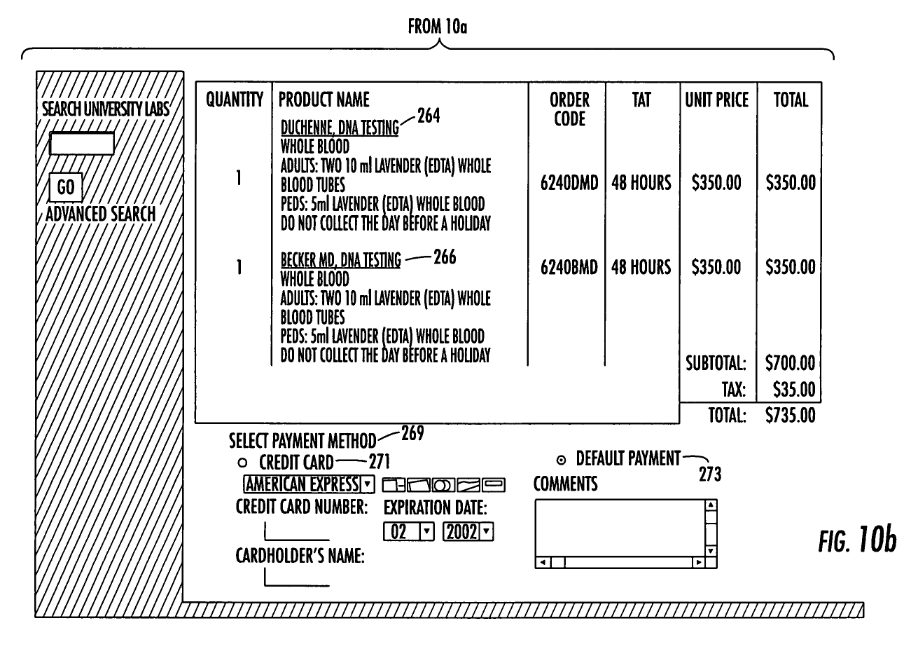

A purchase order software interface 280, FIGS. 10A and 10B, can include vendor information 279 describing the lab or supplier of the test, the billing address 284 of the user, the shipping address 286 of the user, and information about the one or more tests 264, 266 to be purchased. Payment for tests 264, 266 is determined in section 269 of interface 280, in which the user selects button 271 for a credit card or button 273 to select or a default form of payment that the user or server has previously specified. The default form of payment can include billing the hospital directly.

An exemplary algorithm 281, FIG. 11, that can be used with algorithm program 32, FIG. 2, begins at block 283 with an identified symptom, which is polyuria. In block 285, a test is specified, which in this case is confirming the polyuria with a 24 hour urine collection. Test data is obtained in block 287 to check the urine Osm level of the collected urine. Depending on the level of Osm, the algorithm either proceeds to block 288, 290 or 292. For each of blocks 288, 290 and 292, the next block in the algorithm is to perform another test of checking the serum sodium at either block 294 or 296. Depending on the levels of serum sodium and Osm in the urine, the algorithm proceeds to block 298, 300, 302 or 304. If the level of serum sodium brought algorithm 280 to block 298, this would indicate in block 306 a diagnosis of primary polydipsia. If the level of serum sodium brought algorithm 280 to block 300, this would indicate in block 308 a diagnosis of diabetes insipidus. If the levels of serum sodium and Osm brought algorithm 280 to block 302, the next block in algorithm 280 is to measure in the urine the levels of NaCl, HCO₃ ketones. Depending on the level of these substances, algorithm 280 would proceed to either block 312, 314 or 316 which would lead to the specific diagnosis in the appropriate block at 318, 320 or 322. If the level of serum sodium brought the algorithm to block 304, the urine is measured for solutes such as glucose or urea. Depending on the level of these substances, algorithm 280 proceeds to either of blocks 324, 326 or 328 which would then lead algorithm 280 to the appropriate diagnosis in block 330, 332 or 334. The appropriate diagnosis obtained from algorithm 280 is presented to the user as either a provisional, differential or final diagnosis for the caregiver to consider.

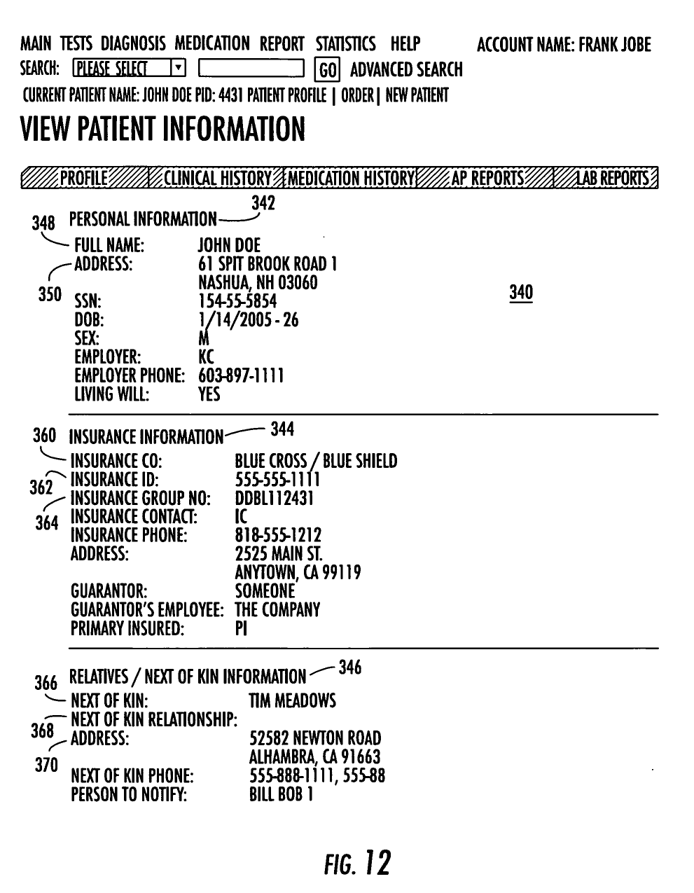

A patient profile software interface 340, FIG. 12, includes information such as personal information 342, insurance information 344, and relatives or next-of-kin information 346. Personal information 342 includes the patient name 348, the patient's address 350 and other personal information about the patient. Insurance information 344 includes the insurance company 360, the insurance identification 362, the insurance group number 364, and other relevant information about the patient's insurance coverage or provider. Relatives or next-of-kin information 346 can include the next of kin 366, the next-of-kin relationship 368, the next-of-kin address 370 and other information relating to the patient's relative or next of kin.

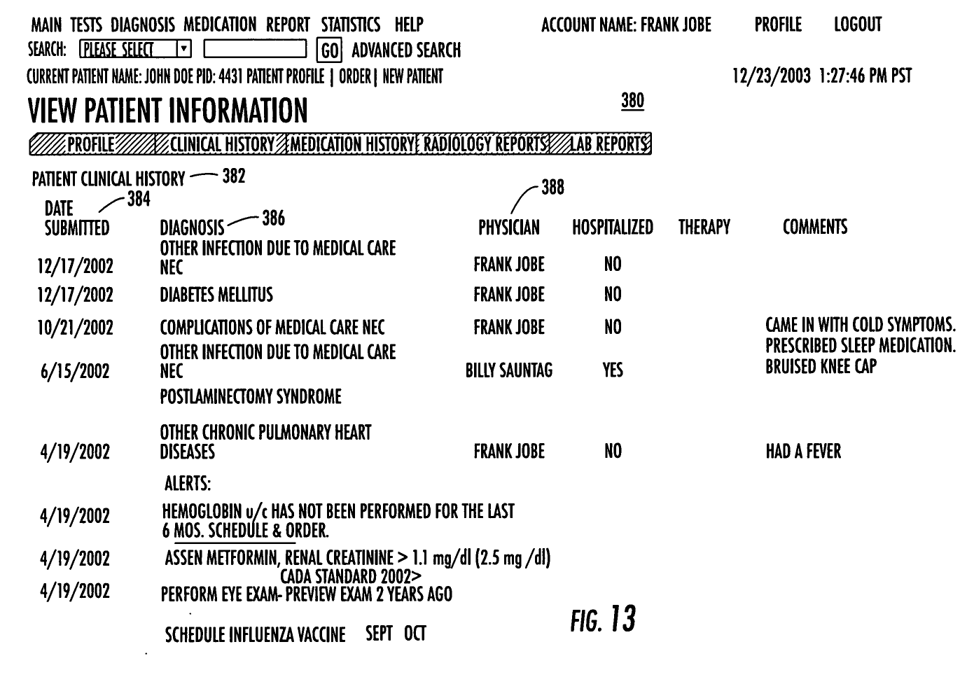

A patient clinical history software interface 380, FIG. 13, displays patient clinical history 282 including the date of entry of the clinical history 384, the diagnosis 386, the physician 388 and other information relevant to the patient's clinical history. A user, such as a physician, can view this information to determine past clinical care that a specific patient has received. Software interface 380 may also include a patient's vital signs and the physician's statistics.

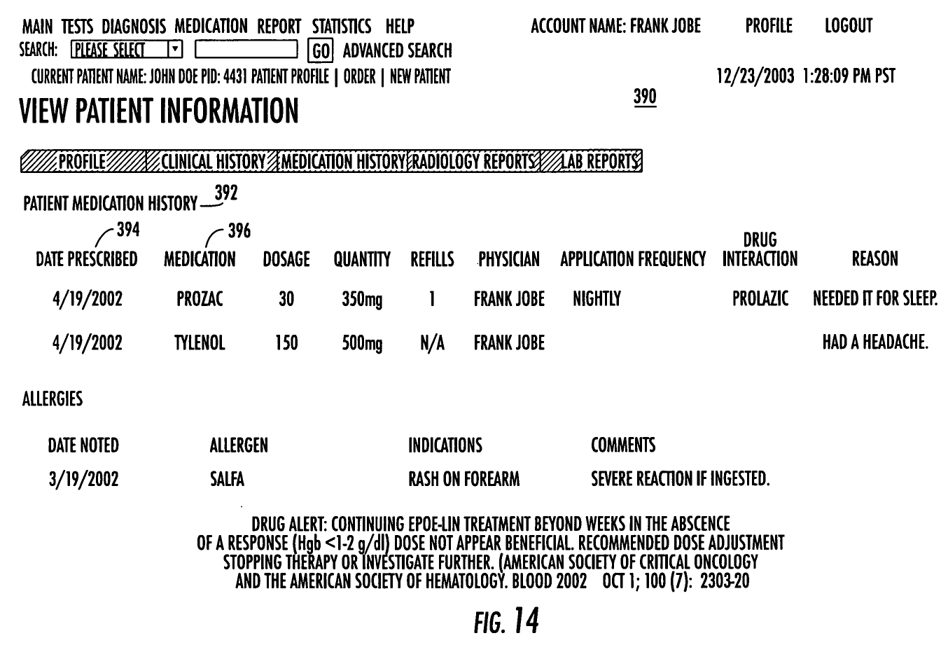

A patient medication history software interface 390, FIG. 14, displays patient medication history 392, which includes the date that medication was prescribed 394, medication prescribed 396 and other relevant information about the patient's medication history. A physician can view this information to determine what medications a patient had been or is currently taking.

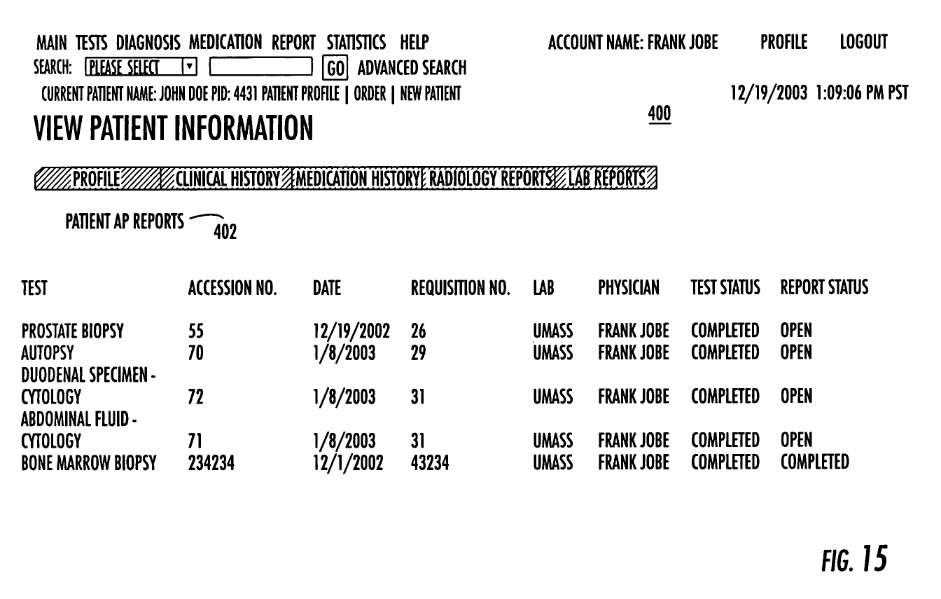

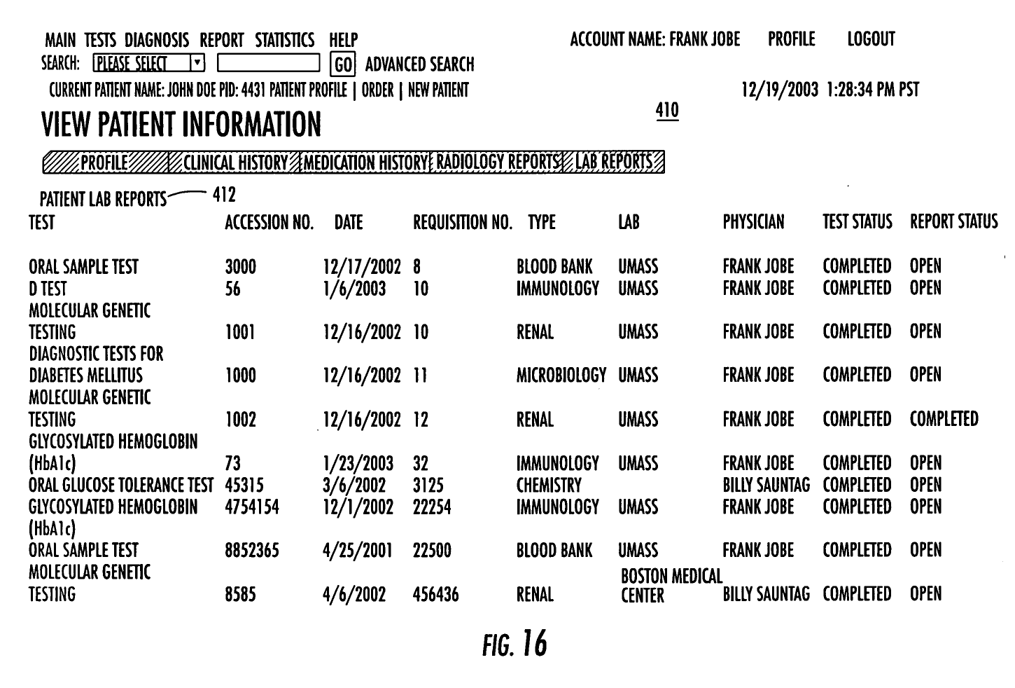

A patient anatomical pathology (AP) report software interface 400, FIG. 15, displays patient AP reports 402 and the relevant information about these reports. A clinical pathology (CP) report software interface 410, FIG. 16, displays patient CP reports 412 and the information related to the CP reports previously obtained.

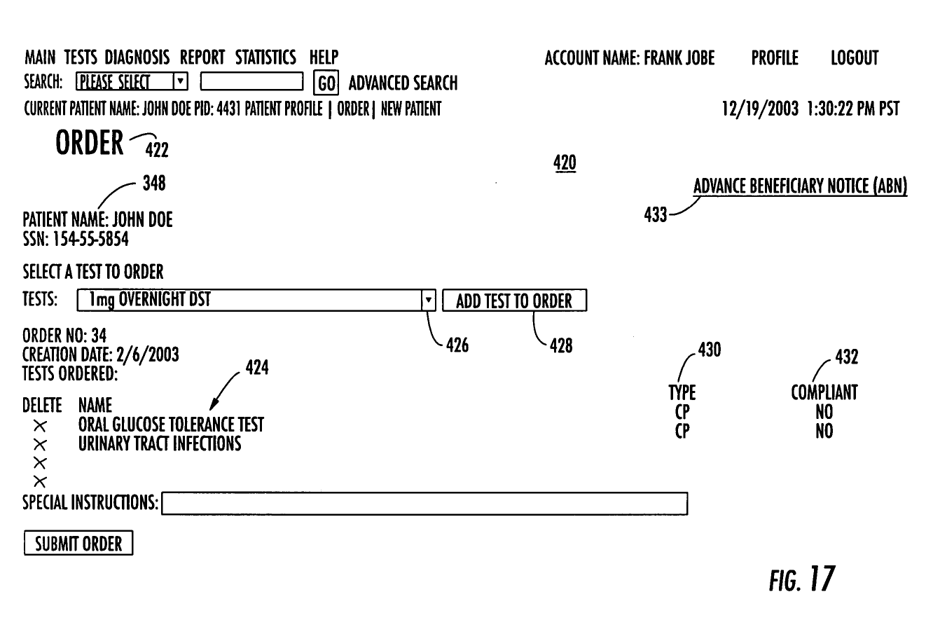

A test ordering software interface 420, FIG. 17, includes a mechanism for ordering one or more tests for a specific patient. Software interface 420 includes the patient's name 348 and the one or more tests 424 ordered for the patient. A user, such as a physician, orders an additional test by selecting button 426, scrolling to the desired test and clicking on the desired test, and selecting button 428 to add the tests to the order. Software interface 420 can include the type 430 of ordered test and whether or not each test is compliant or has constraints 432 against ordering the test. A link 433 is provided to allow a user, such as a patient, to obtain and execute an ABN form. The server can provide the ABN form link 433 to the user if one of the selected tests is designated as having a constraint as at 432.

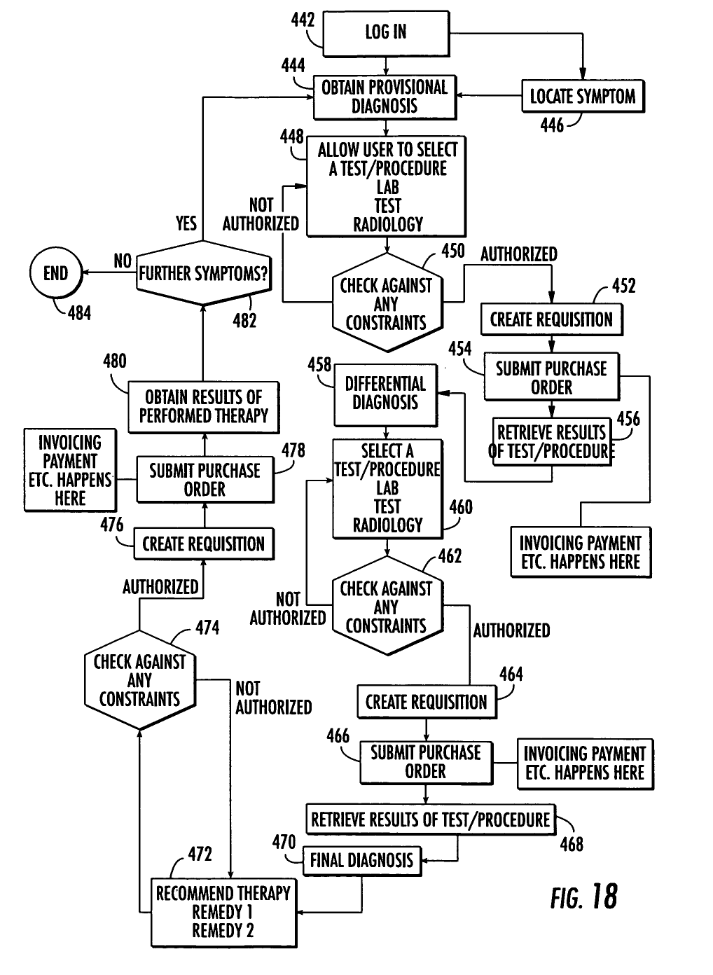

In yet another embodiment, the flowchart of FIG. 18 describes a general or industrial method and begins at step 442 with allowing a user to log in to server 12, FIG. 1, and obtaining a provisional diagnosis from the user in step 444, FIG. 18. The server can either provide the provisional diagnosis based on one or more symptoms given by the user in step 446 or can use an algorithm to provide a diagnosis based upon previously obtained data. In step 448, the server allows the user to select one or more tests or procedures such as a lab procedure, a test or a radiology procedure. In step 450, the server determines whether there are any constraints against ordering the one or more tests/procedures ordered in step 448. If there are any constraints against ordering a test, the user is prompted again to select one or more tests. If there are no constraints against ordering the tests the server creates a requisition in step 452 and prepares a purchase order in step 454. The server obtains payment for the test in step 454. In step 456, the server retrieves the results of the tests. In step 458, the server uses one or more algorithms to determine a differential diagnosis based upon the test results. In step 460, the user is prompted whether or not the user would like to select an additional test. In step 462, the server determines whether there are any constraints against the one or more tests ordered in step 460. If no constraints exist on ordering the test, the server creates a requisition in step 464 and a purchase order in step 466. The server obtains payment for the test in step 466. Once the tests have been completed, the server also obtains the results of the test in step 468 and determines a final diagnosis in step 470 using one or more algorithms.

The server recommends one or more therapies in step 472 based on the final diagnosis determined in step 470. In step 474, the server determines whether there are any constraints against obtaining the recommended therapy and allows the user to select another recommended therapy in step 472 if there are any constraints. In step 476, if the user is authorized to obtain the one or more selected therapies, the server creates a requisition in step 476, submits a purchase order and obtains payment in step 478. In step 480, the server obtains data about the performed therapy and determines in step 482 whether any further symptoms of the condition exist. If it is determined in step 482 that no further symptoms of the condition exist, then the method ends at step 484. If further symptoms of the condition exist then the user can begin the method again at step 444 by obtaining a provisional diagnosis and selecting one or more tests in step 448 to diagnose the condition.

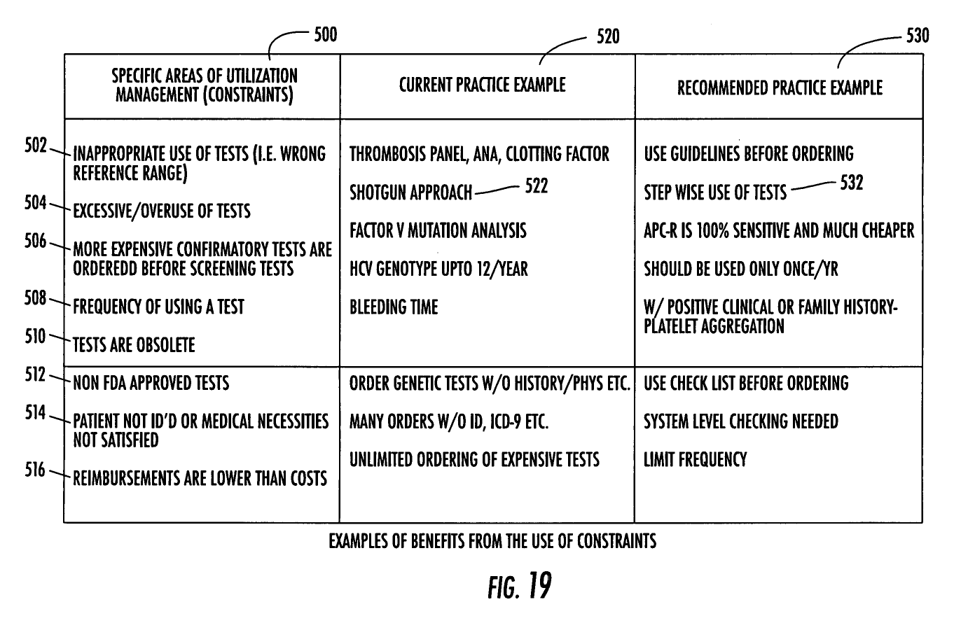

As noted above in steps 100, 148 and 181 of FIGS. 4-6, respectively, a constraint may be applied to prevent the ordering of non-preferred tests or procedures. Examples of constraints 500, FIG. 19, include inappropriate selection of tests 502 such as a test that is in the wrong reference range of a result of a given test/lab/instrument combination, excessive or overuse of tests 504, more expensive confirmatory tests are ordered before screening tests 506, frequency of using a test 508, a test is obsolete 510, a test is not FDA approved 512, a patient is not identified or medical necessities are not satisfied 514, and reimbursements are lower than the cost of a test 516. Column 520 provides examples of a current medical practice that would raise one of the constraints listed in column 500. For example, if a physician uses a shot-gun approach 522 in selecting tests and orders a specific test too frequently, the constraint that the test has been overused 504 may be applied. Column 530 provides recommended practice examples for selecting and ordering tests that will avoid the application of constraints 500. For example, rather than using shot-gun approach 522 in selecting tests, a physician would be advised to the step wise use of tests 532 when selecting a test.

Additionally, a constraint can indicate whether a different lab test or radiology test is preferable, or whether a drug the patient is currently taking will cause an interaction with the selected test. The methods described herein can also include the determination of whether or not to start or stop taking a drug, or to select a drug alternative including a generic versus a branded drug.

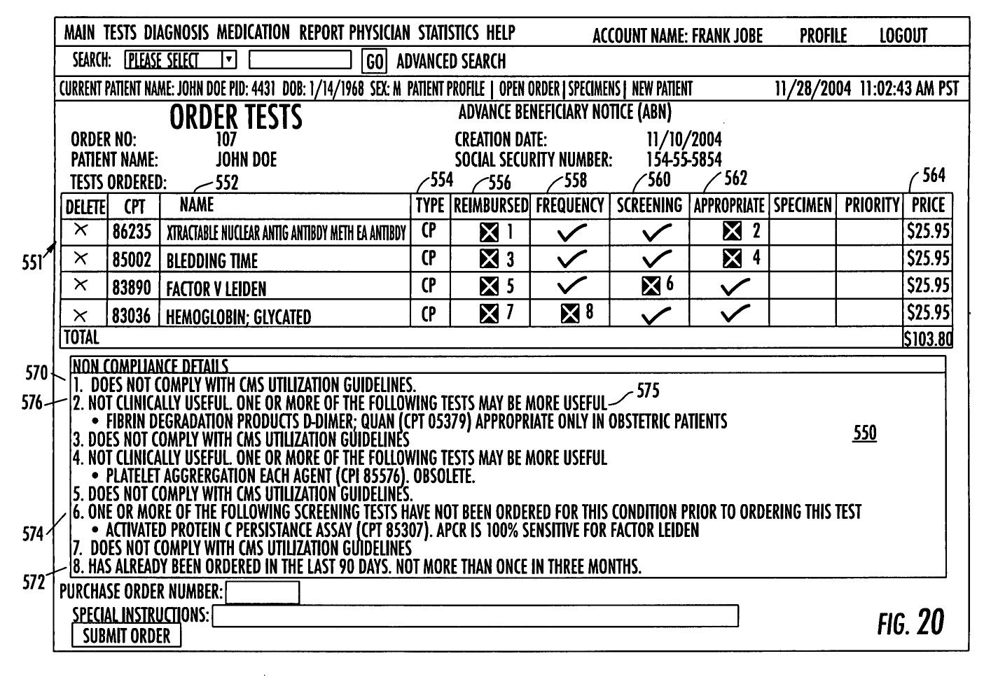

Screen 550, FIG. 20, includes columns 554-564 having information about selected tests in rows 551, such as columns for the name of the test 552, the type of a test 554, whether or not it can be reimbursed 556, whether the frequency of use is appropriate 558, whether proper screening of tests has been completed 560, whether a selected test is appropriate 562, and the price of the test 564. Columns 556-562 include information about constraints that may indicate that a selected test is not appropriate. For example, a constraint may indicate in column 556 that a test will not be reimbursed because it does not comply with CMS utilization guidelines 570. A constraint may indicate in column 558 that a test has been ordered too frequently because the test has already been ordered within a predetermined time, such as within the last ninety days 572. The constraint about screening in column 560 may indicate that one or more screening tests have not been ordered prior to ordering the selected test 574. The constraint in column 562 about the appropriateness of an ordered test may indicate that a test may not be clinically useful 576. Screen 550 may also indicate one or more tests that may be useful 578 if a test is not clinically useful 576.

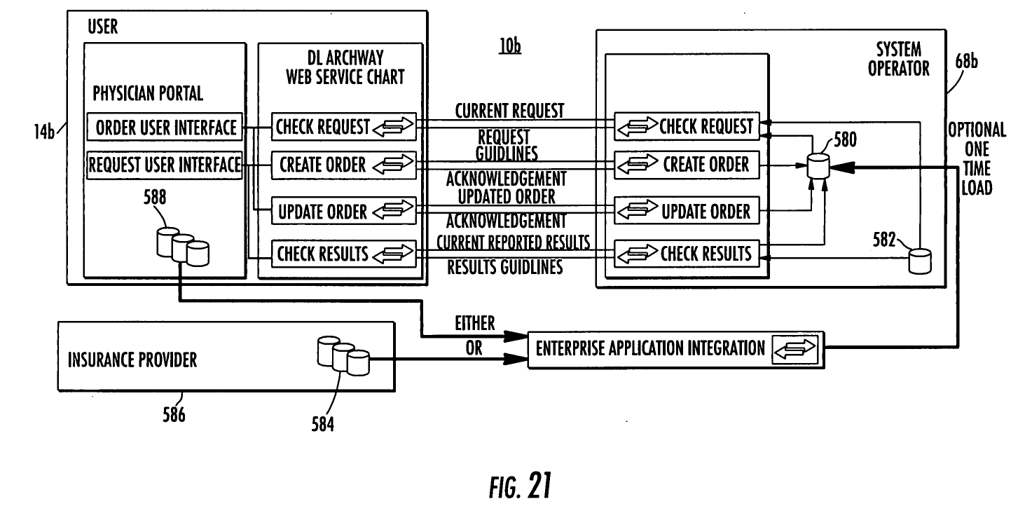

Another embodiment of medical healthcare services system 10b, FIG. 21, includes patient historical database 580 and decision support database 582 located at system operator 68b. Patient historical database 580 includes relevant information necessary to treat a patient and select appropriate tests for the patient if necessary. Decision support database 582 includes information about a plurality of tests and information about the constraints associated with each of those tests, such that system operator 68b can assist user 14b in selecting and ordering tests. Information stored on patient historical database 580 may be obtained from patient historical database 584 located at insurance provider 586 or from patient historical database 588 located at user 14b. The information stored on patient historical database 580 may be loaded only once from patient historical databases 584 or 588 or may be updated more than one time.

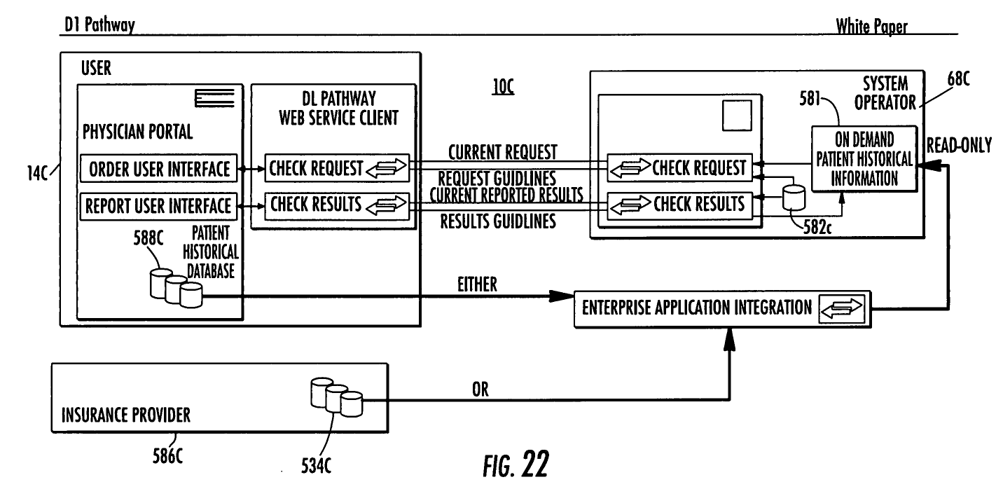

Another embodiment of medical healthcare services system 10c, FIG. 22, does not include a patient historical database as system operator 68c, but rather obtains information about a patient's history from patient historical database 584c located at insurance provider 586c or from database 588c at the location of user 14c. With medical healthcare services system 10c, patient historical information 581 is obtained on demand from either database 584c or 588c and information from these databases is preferably read-only. Patient historical databases 584 and 584c may include one or more databases and may be situated at more than one location.

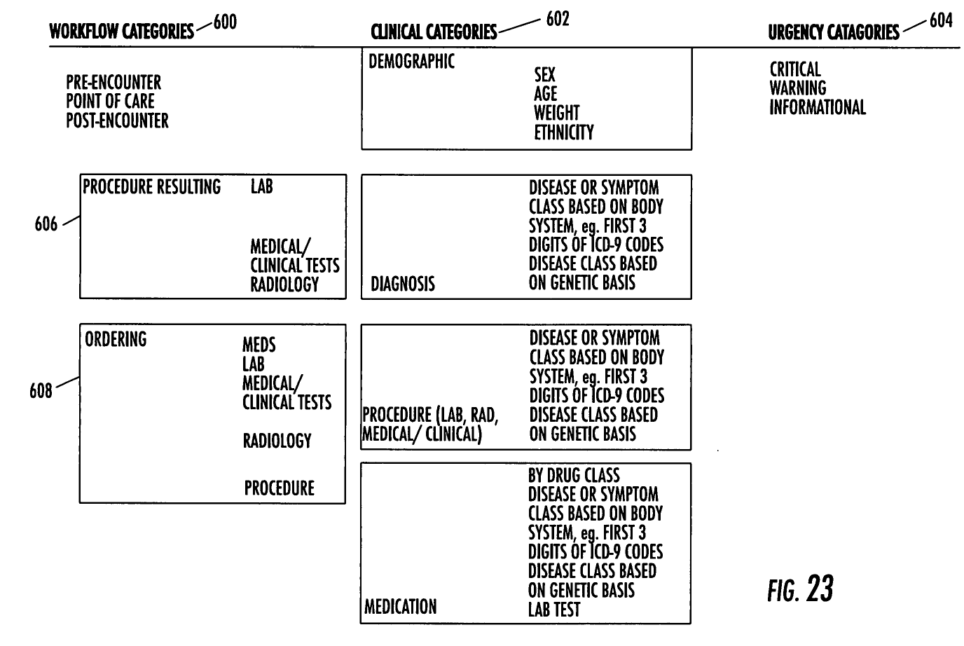

The rules that determine whether a constraint exists can be divided into categories and subcategories as shown in FIG. 23. Categories may include workflow category 600, clinical category 602 or an urgency category 604. Each category may be further divided into subcategories such as procedure resulting 606 or ordering 608. An advantage of dividing the rules into categories or subcategories is that system 10a may expedite the process of determining whether a constraint exists by only reviewing the one or more categories that include the most relevant rules. This division of rule categories can be especially pertinent considering that there may be as many as 15,000 rules in determining whether a constraint exists, which can be time-consuming for a computer system to review.

One of the advantages of the subject invention is that it contributes to the diagnosis, treatment and prevention of HIV/AIDS. For example, if an HIV/AIDS diagnosis is suspected, the subject invention will recommend a screening HIV antibody test. If the result is positive, the subject invention will then recommend a HIV viral load test (CPT code 87536) and a HIV genotype test (CPT code 87901). If these tests are positive, the subject invention will then recommend that a clinical evaluation be performed to confirm the HIV diagnosis. If HIV is confirmed and CD4 counts (from viral load test) are below 350, the subject invention alerts the physician to the treatment of combination therapy. In this case, the subject invention will also provide the physician with the following information:
Single antiretroviral drug therapy does not demonstrate potent and sustained antiviral activity and should not be used. The rare exception, though controversial, is the use of zidovudine monotherapy to prevent perinatal HIV-1 transmission in a woman who does not meet clinical, immunologic, or virologic criteria for initiation of therapy and who has an HIV ribonucleic acid (RNA) <1,000 copies/mL. Most clinicians, however, prefer to use a combination regimen in the pregnant woman for the management of both the mother's HIV infection and in the prevention of perinatal transmission. The efficacy of zidovudine monotherapy during pregnancy to reduce perinatal transmission was identified in the Pediatric AIDS Clinical Trial Group (PACTG) 076 study. The goal of therapy in this case is solely to prevent perinatal HIV-1 transmission. Zidovudine monotherapy should be discontinued immediately after delivery. Combination antiretroviral therapy should be initiated postpartum if indicated.

The methods of the present invention can be performed with a server or computer and computer software installed thereon that has instructions to perform the steps of the invention. Alternatively, methods of the present invention can be performed with equipment that has installed hardware or firmware having instructions to perform the steps of the invention. Software used with embodiments of the present invention can be stored on computer readable medium for storing data, such as, for example, but not limited to, floppy disks, magnetic tape, zip disks, hard drives, CD-ROM, optical disks, RAM, ROM, PROM or a combination of these.

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments.

What is claimed is:

1. A method for selecting over a network one or more tests for a medical condition for a patient, the method comprising:
providing to the user over the network one or more tests for the patient that can be selected;
allowing the user to select over the network one or more tests;
determining whether a constraint exists on ordering any of the selected tests;
ordering the selected tests over the network;
obtaining a result of each of the ordered tests; and
providing an automated evaluation based upon feedback resulting from the ordered tests.

2. The method of claim 1 further including providing a report based upon the automated evaluation.

3. The method of claim 1 in which the step of determining whether a constraint exists includes evaluating whether a different lab or radiology test is preferable.

4. The method of claim 1 in which the step of determining whether a constraint exists includes evaluating whether a drug the patient is currently taking will cause an interaction with the selected test.

5. The method of claim 4 further including the step of determining whether to start or stop a drug, to select drug alternatives, or to change a drug dosage.

6. The method of claim 1 in which the step of providing an evaluation includes providing an evaluation of the one or more tests provided to the user.

7. The method of claim 1 in which the step of providing an evaluation includes providing an evaluation of the constraints of diagnostic algorithms.

8. The method of claim 1 in which the step of providing an evaluation includes providing an evaluation of the user.

9. The method of claim 1 in which the feedback is obtained from the users.

10. The method of claim 1 in which the feedback is obtained from outcome data of the ordered tests.

11. The method of claim 1, further including the steps of:
recommending to the user over-the network one or more tests based on a provisional diagnosis of the condition; and
providing to the user over the network an analysis of the one or more recommended tests if more than one test is recommended.

12. The method of claim 1 further including the steps of:
recommending over the network at least one treatment based on the test results to cure the condition;
identifying whether there is any constraint on ordering the recommended treatment; and
ordering the treatment if no constraint exists for user to obtain the treatment in which the treatment is selected from a medical or surgical procedure, one or more drugs, or a combination thereof.

13. The method of claim 12 in which each of the constraints have a plurality of levels of significance.

14. The method of claim 1 further including the step of allowing the user to provide an override to order the test if a constraint exists on ordering the test.

15. The method of claim 14 further including the step of providing a notification of the override to one or more parties.

16. The method of claim 1 in which the step of determining whether any constraints exist includes comparing a code for the provisional diagnosis with a code for each of the selected tests to determine whether there is any constraint on each of the selected tests.

17. The method of claim 1 further including the step of providing to the user a cost analysis of each of the tests if more than one test is recommended.

18. The method of claim 1 in which the user is a physician.

19. The method of claim 1 in which the step of determining whether any constraints exist includes determining whether a test meets guidelines, is ordered too frequently, is obsolete, is FDA approved, is too expensive, or would be ineffective due to a drug the patient is taking.

20. The method of claim 1 in which a constraint is selected from the group of causes of increased follow-up visits, a drug the patient is taking, a drug the patient is not taking, the result of a screening test, an indication or the lack of an indication from a test result or prior diagnosis.

21. A method for selecting over a network one or more tests for a condition for a patient, the method comprising the steps of:
allowing a user to select over the network a first test for the patient;
determining whether a constraint exists on ordering the selected first test;
recommending to the user over the network one or more secondary tests if a constraint exists to obtain the selected first test;
allowing the user to select over the network one of the secondary tests;
ordering the selected secondary test over the network; and
obtaining a result of the selected secondary test.

22. The method of claim 21 in which the step of determining whether a constraint exists includes evaluating whether a test meets guidelines, is ordered too frequently, is obsolete, is FDA approved, is too expensive, or would be ineffective due to a drug the patient is taking.

23. The method of claim 21 further including the steps of:
recommending to the user over the network one or more initial tests based on a provisional diagnosis of the condition; and
providing to the user over the network an analysis of the one or more recommended initial tests if more than one initial test is recommended.

24. The method of claim 21 further including the step of allowing the user to provide an override to order the selected first test if the test is not covered by insurance.

25. A system for providing medical healthcare services, comprising:
a computer readable medium having computer readable program code for ordering over a network one or more tests for a condition, the computer readable program code executable on a computer system and including instructions for:
causing the computer system to allow a user to select over the network one or more of the tests,
causing the computer system to determine whether a constraint exists on ordering any of the selected tests,
causing the computer system to order the selected tests over the network,
causing the computer system to obtain a result of each of the selected tests, and
causing the computer system to provide an evaluation based upon feedback resulting from the ordered tests.

26. The system of claim 25 in which the computer readable program code further includes instructions for causing the computer system to obtain payment for a test if a constraint exists on ordering the test.

27. The system of claim 25 in which the computer readable program code further includes instructions for causing the computer system to provide one or more final diagnoses for the condition based on the result of each of the selected tests.

28. The system of claim 25 in which the computer readable program code further includes instructions for causing the computer system to allow the user to provide over the network an override to order the test if a constraint exists on ordering the test.

29. The system of claim 28 in which the computer readable program code further includes instructions for causing the computer system to provide a notification of the override to one or more parties.

30. The system of claim 25 in which the computer readable program code for causing the computer to provide an evaluation based upon feedback includes computer readable program code for causing the computer system to provide an evaluation of the one or more tests provided to the user.

31. The system of claim 25 in which the computer readable program code for causing the computer to provide an evaluation based upon feedback includes computer readable program code for causing the computer system to evaluate whether a drug the patient is currently taking will cause an interaction with the selected test.

32. The system of claim 25 in which the computer readable program code for causing the computer to provide an evaluation based upon feedback includes computer readable program code for causing the computer system to provide an evaluation of the one or more tests provided to the user.

33. A server for providing medical healthcare services, the server comprising:
a computer including a processor and computer readable program code executable on the processor for ordering over a network one or more tests for a condition, the computer readable program code configured to:
allow a user to select over the network one or more of the tests,
determine whether a constraint exists on ordering any of the selected tests,
order over the network the selected tests,
obtain a result of each of the selected tests, and
provide an evaluation based upon feedback from the ordered tests.

34. The server of claim 33 in which the computer readable program code is further configured to obtain payment for a test if a constraint exists on ordering the test.

35. The server of claim 33 in which the computer readable program code is further configured to allow the user to provide an override to order a test if a constraint exists on ordering the test.

36. The server of claim 33 in which the computer readable program code is further configured to provide a notification of the override to one or more parties.

37. The server of claim 33 in which the computer readable program code is further configured to cause the computer system to provide a notification of the override to one or more parties.

38. The server of claim 33 in which the computer readable program code for causing the computer to provide an evaluation based upon feedback includes computer readable program code for causing the computer system to provide an evaluation of the user or the one or more tests provided to the user.

39. The server of claim 33 in which the computer readable program code for causing the computer to provide an evaluation based upon feedback includes computer readable program code for causing the computer system to evaluate whether a drug the patient is currently taking will cause an interaction with the selected test.

40. The server of claim 33 in which the computer readable program code for causing the computer to provide an evaluation based upon feedback includes computer readable program code for causing the computer system to provide an evaluation of the one or more tests provided to the user.

41. The server of claim 33, further including a database that includes information about patients' historical data.

42. The server of claim 33, further including a database that includes information about decision support guidelines.

43. The server of claim 33 in which the computer readable program code is further configured to:
collect user feedback; and
modify the recommended tests based upon the user feedback.

44. A method for selecting over a network one or more procedures for a medical condition for a patient, the method comprising:
providing to the user over the network one or more procedures for the patient that can be selected;
allowing the user to select over the network one or more procedures;
determining whether a constraint exists on ordering any of the selected procedures;
ordering the selected procedures over the network;
obtaining a result of each of the ordered procedures; and
providing an automated evaluation based upon feedback resulting from the ordered procedures.

45. The method of claim 44 in which the step of determining whether a constraint exists includes the step of reviewing a plurality of rules.

46. The method of claim 45 in which the rules are divided into categories of rules.

47. The method of claim 46 in which the step of reviewing the plurality of rules includes reviewing only some of the categories of rules to expedite the step of determining whether a constraint exists.

48. The method of claim 44 in which the procedure is a test and the test includes a screening HIV antibody test.
